(19) United States
(12) Patent Application Publication
(10) Pub. No.: US 2012/0109041 A1
(43) Pub. Date: May 3, 2012

(54) APPLICATOR DEVICE FOR COSMETIC AND/OR MEDICAL USE

(76) Inventor: Jutta Munz, Munchen (DE)

(21) Appl. No.: 13/262,573

(22) PCT Filed: Mar. 25, 2010
(86) PCT No.: PCT/DE2010/000342

§ 371 Date: Jan. 4, 2012

(30) Foreign Application Priority Data
Mar. 30, 2009 (DE) 10 2009 014 976.7

(51) Int. Cl. A61M 35/00; A45D 34/04

(52) U.S. Cl. 604/20; 604/22; 401/1

(57) ABSTRACT

The invention relates to an applicator device, comprising an applicator element (9) for applying a substance (27) present in a substance chamber (25, 27) to a skin section. The applicator device (1) comprises an applicator part (2) having an upper housing part (8) and a vibration part (3) having a lower housing part (4), wherein in the lower housing part (4) a vibration element (10) is provided which can be actuated by a power source (6) and the vibrations of which can be transmitted to the upper housing part (8) and the applicator element (9), and wherein an activator device (22) for generating heating or cooling energy is provided in the upper housing part (8), wherein the heating or cooling energy can be transferred to the substance (27) present in the substance chamber (25, 27).

The present invention relates to an applicator device for cosmetic and/or medical use.

It is known to cool a person's skin for medical and/or cosmetic reasons. US 2006/0067962 A1 discloses a skin-cooling means which comprises components which make it possible both to cool the skin rapidly and to cool the skin over a relatively long period. For cooling the skin, this known means basically comprises water, alcohol, a surfactant and water-soluble silicone. US 2006/0067962 A1 discloses an applicator in which the skin-cooling means is arranged on a substrate. In this case, the substrate is attached in the form of a plaster to a corresponding portion of skin.

WO 03/000089 A2 describes a disposable applicator device with which in particular cosmetic liquids or creams can be applied to a skin surface to be treated of a human body. In this case, the liquid is stored in a storage layer and dispensed onto an applicator surface via a liquid-controlling control layer. An impermeable layer is also provided which prevents the liquid from flowing off the applicator surface.

DE 199 42 566 A1 describes a cooling pad for use in cooling processes, which cooling pad contains a salt mixture mixed with a gelling agent and keeps this mixture separated from an activator inside the cooling pad until they are brought together. When the activator, which may be water, is added to the salt mixture mixed with the gelling agent, this results in a cooling reaction in which the temperature of the cooling pad falls to approximately 0° C.

WO 2004/043534 A1 discloses an applicator device comprising an applicator element for applying a substance contained in a substance chamber to a portion of skin. In this case, the applicator part can contain a vibrator and a means with which the skin can be cooled or heated.

The object of the present invention is to configure an applicator device in such a way that a cosmetic and/or medical substance which is dispensed onto a part of a person's skin or body requiring care or treatment is applied in the form of a cooling or heating substance, a gentle vibration massage occurring simultaneously to stimulate lymphatic activity.

This object is achieved by an applicator device having the features of claim 1.

The main advantage of the present invention is that portions of a person's skin requiring care or treatment, in particular the eye region or for example a part of the body affected by cellulite or pain or tension, are made to vibrate by a gentle vibration massage to stimulate lymphatic activity and/or blood flow, any possible swelling of said skin portions decreasing. At the same time a substance which has a nurturing and/or medicinal effect (for example which reduces swelling, promotes blood flow or has a draining or dehydrating effect) is advantageously applied to the skin portions in question via the applicator device according to the invention. Because said substance is applied to a skin portion with a simultaneous vibration effect, particularly effective and fast-acting skin care is possible. It is particularly advantageous that the medicinal and/or cosmetic substance can be applied in a cooled or heated form. As a result, the effect of the substance and thus also the physiological skin and body reactions can be improved and accelerated. It is also conceivable to use the applicator device 1 according to the invention for treating orthopaedic conditions or for preferably post-operative treatment of scars and tissue and in connection with other indicators, for example palliatively for pain relief or in the case of inflammatory diseases.

A further basic advantage of the present invention is that the applicator device is configured in such a way that the applicator part containing the substance to be applied is formed as a disposable part, in such a way that the vibration part of the applicator device according to the invention can be used selectively for different substances. In this case, the disposable part can simultaneously also comprise the means causing cooling or heating. For example, these means may be reactants which are known per se.

In an advantageous configuration of the present invention there is also the possibility of light therapy. Within the context of a NASA research programme, it has been proved in particular that light therapy, in which light is preferably produced using light emitting diodes (LEDs), can accelerate the healing process of wounds and the growth of human tissue (cell generation). In addition, it has been found that the light energy emitted by the LEDs improves the metabolism of the cells and accelerates cell regeneration. Light therapy—not least owing to its uplifting effect—is thus an effective method for the treatment and prophylaxis of many illnesses.

Advantageous configurations of the invention emerge from the dependent claims.

The invention and its configurations are described in detail below in conjunction with the figures, in which:

FIG. 1 schematically shows a side view of the applicator device according to the invention;

FIG. 2 schematically shows a longitudinal section through the applicator device according to the invention of FIG. 1;

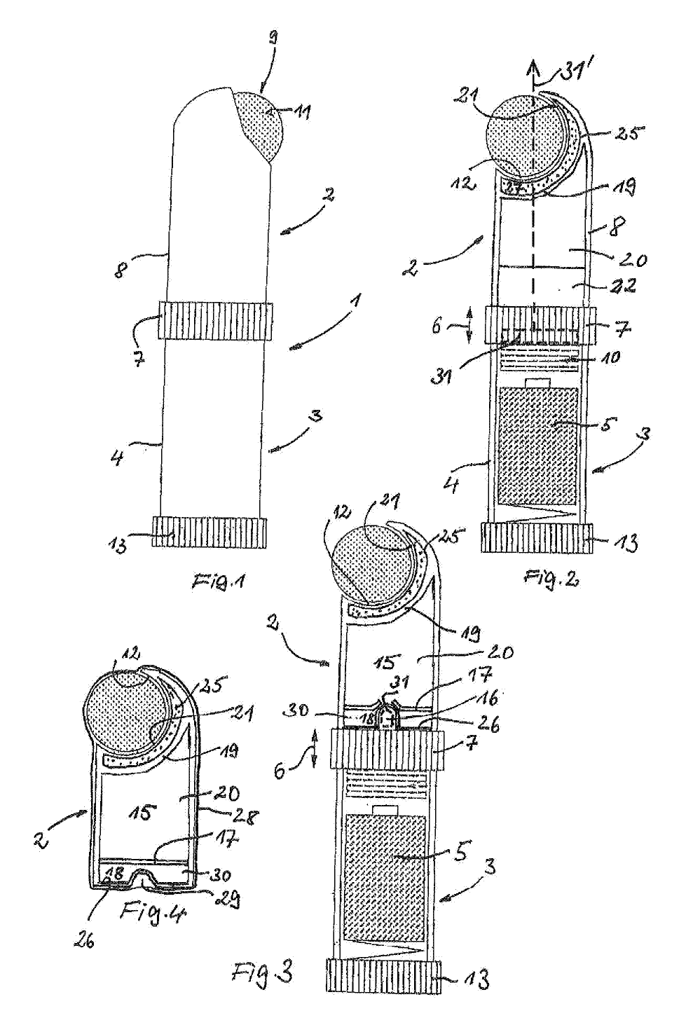

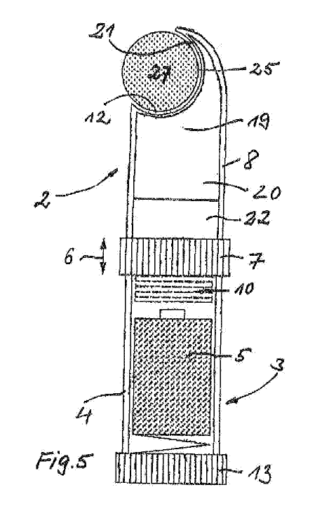

FIGS. 3 and 4 are schematic drawings illustrating the principle of an activator device and

FIG. 5 shows a development of the invention.

In the manner which can be seen in particular in FIGS. 1 and 2, the present applicator device 1 basically consists of an applicator part 2, which comprises a substance 27 to be applied and the means causing the cooling or heating of this substance 27, and a vibration part 3. According to FIG. 2, the vibration part 3 basically comprises, in a lower housing part 4, an energy source 5, preferably in the form of a battery, a control part 13 which is preferably arranged at the lower end of the housing part 4 and comprises at least an on/off switch, a vibration element 10 which produces oscillations in the longitudinal direction of the vibration part 3 (see arrow 6) and which is supplied with current by the energy source 5, and a connecting part 7, preferably arranged on the upper side of the housing part 4, for connecting the upper housing part 8 to the lower housing part 4. The oscillation amplitude of the vibrations of the vibration element 10 can preferably also be adjusted on the control part 13. The vibration element 10 can for example be a piezo component. It should be noted that the energy source 5 can also be formed via the possibility of a connection to an alternating or direct voltage network.

The applicator part 2 which can be attached to the vibration part 3 using the connecting part 7 basically comprises, in the upper housing part 8, an applicator element 9 via which a medicinal and/or cosmetic substance to be applied to a portion of skin can be applied to said portion of skin. For example, the applicator element 9 is a “roll-on” element in the form of a spherical part 11 which dispenses the substance via its surface and is known from the conventional “deo-rollers”. The spherical part 11 is contained in a corresponding, approximately hemispherical trough 12 of the upper housing part 8. Inside the upper housing part 8 there is a substance chamber 25 containing the substance 27 to be applied, from which substance chamber the substance is transferred to the surface of the spherical part 11 during use of the applicator device 1. The spherical part 11 is set in rotation via sliding movements on the skin and dispenses the substance 27 transferred from the substance chamber 25 onto the portion of skin. A roller-shaped part forming the applicator element 9 can also be provided instead of the spherical part 11.

With reference to FIG. 3, the substance chamber 25 is separated by means of a wall part 19 from a reaction chamber 20 in which cooling energy for cooling the substance 27 or heat for heating the substance 27 is produced during use of the applicator device 1, as described in detail below. The substance 27 is dispensed to the spherical part 11 in a manner known per se via the trough wall 21 limiting the trough 12. In order to prevent the substance 27 from drying out in the region of the applicator element 9, allow re-use and ensure the required hygiene and protection of the activator 22, the entire applicator part 2, preferably configured as a disposable part, is preferably contained for distribution in a cover 28 which is impermeable to moisture, is only opened during use of the applicator part 2 and is distanced therefrom (see FIG. 4). The cover 28 can also be used for advertising and decorative purposes.

The means required for producing cooling energy or heat are located in the reaction chamber 20 adjacent to the wall part 19 on the side remote from the substance chamber 25. In very general terms, this means is a schematically shown activator 18 which is able to produce cooling energy for cooling the substance 27 or heat for heating it.

For example, in the manner known from DE 199 42 566 A1, a salt mixture 15, which is mixed with a gelling agent and is separated by a dividing wall 17 from a chamber 28 containing an activator 18 in the manner shown in FIG. 4, is located in the reaction chamber 20 as a first means for producing cooling energy. The activator 18 forms a second means. For example, the activator 18 consists of water. The gelling agent can consist of methyl cellulose sodium. When the two means 15 and 18 separated from one another by the dividing wall 17 are brought together, the cooling energy required to cool the substance 27 immediately at the wall part 19 is produced.

In order to bring the two means 15 and 18 together, the dividing wall 17 is preferably severed automatically when the two housing parts 4 and 8 are connected using the connecting part 7. For this purpose, a pin part 16 is arranged on the connecting part 7 or on the lower housing part 4, which pin part is expediently arranged centrally on the connecting part 7 and projects beyond the connecting part 7 towards the dividing wall 17. When the lower housing part 4 is connected to the upper housing part 8, the pin part 16 enters, in the manner shown in FIG. 4, a recess 29 in a base 26 limiting the upper housing part 8 and the chamber 30 from the lower housing part 4. The base 26 and the recess 29 are configured to be so flexible that when the housing parts 4 and 8 are connected a pressure applied by the pin part 16 acts on the dividing wall 26 and causes the dividing wall 17 to break in such a way that the two means 15 and 18 come into contact with each other to produce cooling energy. This is shown in FIG. 3.

The substance 27, preferably a liquid, a cream or a gel, which is contained in the substance chamber 25 has a nurturing, in particular also a swelling reduction effect. Owing to its consistency it facilitates the sliding action of the spherical part 11 and thus optimises for example a gentle lymphatic drainage. In particular, the present applicator device 1 is suitable for highly effective care of the eye regions, which may be swollen for example in the morning or generally in the case of tiredness. In the case of the present applicator device 1, the effect of the nurturing substance 27 is increased by cooling energy or heat which is produced and also by the oscillations which are produced in the vibration part 3 and transferred to the applicator part 2. The applicator device 1 can also be used in the case of headaches, preferably in the region of the temples or forehead. It is also conceivable, in the case of colds or hay fever, to produce heat from the outside in the region of the neck or maxillary sinus in order to achieve a healing effect, in particular in conjunction with the substance 27.

For oscillation transfer, the vibration element 10 is coupled to the upper housing part 8 when the housing parts 4 and 8 are connected. As is known, the connecting part 7 preferably comprises a plug-in, snap or screw connection.

A configuration of the invention in which the applicator part 2 is in the form of a disposable part is particularly advantageous. As a result, different applicator parts 2 having different substances and also different heating or cooling means can selectively be connected to the same vibration part 3.

In the manner indicated in FIG. 2 by way of continuous lines, the present applicator device 1 can comprise a light source 31, the light from which can be radiated onto the skin or body parts to be treated in order to produce therein the effect of promoting wound-healing or cell regeneration or the uplifting effect. In this case, the light source 31 is preferably arranged in the connecting part 7 (FIG. 2) or in the pin part 16 (FIG. 3) or in the vibration part 3. The applicator part 2 and the corresponding parts thereof (for example base 26, activator 22, dividing wall 17, reaction chamber 20, wall part 19, trough wall 21, substance 27, means 15 and 18) and the applicator element 9 are formed so as to be transparent, in such a way that the light ray 31′ produced by the light source 31 can pass unhindered through the applicator part 2 and the applicator element 9 to the skin or body parts to be treated. The light source 31 can be configured in such a way that it can selectively produce light in different colours, depending on the application.

In contrast to the configuration according to FIGS. 2 to 4, it is also conceivable to provide the substance to be applied, in accordance with FIG. 5, in the applicator element 9, in other words in the spherical part 11 or in the corresponding roller-shaped part itself, the applicator element 9 then having a cover consisting of a semi-permeable material. In this way, during use of the applicator device 1 the substance can transfer to the skin or body parts the gentle pressure exerted on the applicator element 2. In this case, depending on the application the surface of said cover can be configured in an amorphous manner or with pimples for promoting blood flow.

### List of Reference Numerals

1 applicator device
2 applicator part
3 vibration part
4 lower housing part
5 energy source
6 arrow
7 connecting part
8 upper housing part
9 applicator element
10 vibration element
11 spherical part
12 trough
13 control part
15 means
16 pin part
17 dividing wall
18 means
19 wall part
20 reaction chamber
21 wall region
22 activator
25 substance chamber
26 base
27 substance
28 cover
29 recess
30 chamber
31 light source
31′ light ray

1. Applicator device comprising an applicator element (9) for applying a substance (27) contained in a substance chamber (25) to a part of the skin or body, characterised in that the applicator device (1) comprises an applicator part (2) having an upper housing part (8) and a vibration part (3) having a lower housing part (4), in that in the lower housing part (4) a vibration element (10) which is operable by means of an energy source (5) is provided, the oscillations of which can be transferred to the upper housing part (8) and the applicator element (9), and in that in the upper housing part (8) an activator device (20, 22) for producing heat or cooling energy is provided, it being possible to transfer the heat or cooling energy to the substance (27) contained in the substance chamber (25).

2. Applicator device according to claim 1, characterised in that the vibration element (10) is a piezoelectric component.

3. Applicator device according to claim 1, characterised in that the applicator element (9) is in the form of a spherical part (11) or a roller-shaped part which is arranged in a trough (12) formed by a trough-shaped wall region (21) of the upper housing part (8), the substance chamber (25) being adjacent to the side of the wall region (21) opposite the spherical part (11) or the roller-shaped part and it being possible to dispense the substance (27) to the spherical part (11) via the wall region (21).

4. Applicator device according to claim 3, characterised in that the substance chamber (25) is formed in the upper housing part (8) by the trough-shaped wall region (21) and a wall part (19) spaced apart from the trough-shaped wall region (21), and in that the activator device (20, 22) is arranged on the side of the wall part (19) remote from the substance chamber (25).

5. Applicator device according to claim 1, characterised in that the applicator part (9) is in the form of a spherical part (11) or a roller-shaped part which is arranged in a trough (12) formed by a trough-shaped wall region (21) of the upper housing part (8), the substance chamber (25) being formed by the interior of the spherical part (11) or of the roller-shaped part and it being possible to dispense the substance (27) via the cover of the spherical part (11) or of the roller-shaped part.

6. Applicator device according to claim 1, characterised in that the activator device (20, 22) comprises a reaction chamber (20) which is adjacent to the substance chamber (25) and is formed in the upper housing part (8) by a dividing wall (17) spaced apart from the substance chamber (25), and in that a chamber (30) is arranged on a side of the dividing wall (17) remote from the reaction chamber (20), a first means (15) being located in the reaction chamber (20) and a second means (18) being located in the chamber (30), which means cooperate and produce cooling energy or heat when the dividing wall is severed.

7. Applicator device according to claim 6, characterised in that the chamber (30) comprises, on the side thereof facing the vibration part (3), a base (26) which is flexible at least in part, is spaced apart from the dividing wall (17) and, when the upper housing part (8) is connected to the lower housing part (4), can be deformed by a pin part (16) of the lower housing part (4) in such a way that the pin part (16) exerts a pressure on the dividing wall (17) in order to pierce or open it so that the first means (15) and the second means (18) come into contact with each other.

8. Applicator device according to claim 1, characterised in that the substance (27) is a liquid, a cream or a gel and has a nurturing and/or swelling reduction and/or cosmetic and/or healing effect.

9. Applicator device according to claim 1, characterised in that the first means (15) is a first chemically reactive substance and in that the second means (18) is a second chemically reactive substance.

10. Applicator device according to claim 9, characterised in that the first means (15) is a salt mixture (15) mixed with a gelling agent, preferably methyl cellulose sodium, and the second means (18) is water.

11. Applicator device according to claim 1, characterised in that the lower housing part (4) comprises a control part (13) having an on/off switch.

12. Applicator device according to claim 11, characterised in that the oscillation amplitude and/or the frequency and/or the oscillation mode of the vibration element (10) can be adjusted on the control part (13).

13. Applicator device according to claim 1, characterised in that the upper housing part (8) and the lower housing part (4) can be interconnected in a releasable manner using a connecting device (7).

14. Applicator device according to claim 13, characterised in that the connecting device (7) is a screw, plug-in, snap or bayonet connection.

15. Applicator device according to claim 12, characterised in that the applicator part (2) is formed as a disposable part.

16. Applicator device according to claim 15, characterised in that the applicator part (2) is arranged in a cover (28) which surrounds the upper housing part (8).

17. Applicator device according to claim 1, characterised in that the energy source is in the form of a battery or an accumulator or is in the form of a terminal which can be connected to an alternating or direct voltage network.

18. Applicator device according to claim 1, characterised in that it comprises a light source (31) which produces light to be radiated onto the skin or body parts to be treated.

19. Applicator device according to claim 18, characterised in that the light source (31) is formed by at least a light emitting diode.

20. Applicator device according to claim 17, characterised in that the light source (31) is arranged in the vibrator part (3), in the connecting part (7) or in the pin part (26) and produces a light ray (31′) which radiates through the transparent applicator part (2) and the applicator element (9).

21. Applicator device according to claim 18, characterised in that the light source (31) is formed for the selective production of light rays (31′) of different colours.

22. Use of the applicator device according to claim 1, for treating pain and/or tension and/or inflammation, and/or for the preferably post-operative treatment of scars and tissue and/or cellulite, and/or for promoting blood flow and/or for stimulating lymphatic activity and/or for drainage and/or for dehydration and/or for treating swelling.
